(19) United States
(12) Patent Application Publication
(10) Pub. No.: US 2010/0136327 A1
(43) Pub. Date: Jun. 3, 2010

(54) Method of preparation of a MWCNT/polymer composite having electromagnetic interference shielding effectiveness

(75) Inventors: Chen-Chi Martin Ma, Hsinchu (TW); Siu-Ming Yuen, Hsinchu (TW); Chia-Yi Chuang, Hsinchu (TW); Kuo-Chi Yu, Hsinchu (TW); Sheng Yen Wu, Hsinchu (TW)

Correspondence Address: BACON & THOMAS, PLLC, 625 SLATERS LANE, FOURTH FLOOR, ALEXANDRIA, VA, 22314-1176, US

(73) Assignee: National Tsing Hua University, Hsinchu (TW)

(21) Appl. No.: 12/654,331

(22) Filed: Dec. 17, 2009

Related U.S. Application Data
(63) Continuation-in-part of application No. 12/081,517, filed on Apr. 17, 2008.

(30) Foreign Application Priority Data
Mar. 10, 2009 (TW) 98107777

(51) Int. Cl. B32B 27/00; C07C 53/00; C07C 43/03; C08K 3/04; B05D 3/00; B32B 38/00; B05D 5/12

(52) U.S. Cl. 428/335; 562/512; 568/671; 523/137; 428/334; 427/385.5; 156/278; 427/117; 977/742; 977/932

(57) ABSTRACT

The present invention provides a modified carbon nanotube having —C(O)—R′ or —C(O)—R—COOH covalently bounded to a surface of carbon nanotube, wherein R′ is C1-C26 alkyl or C2-C26 alkenyl, and R is C1-C26 alkylene or C2-C26 alkenylene. The present invention also discloses a carbon nanotubes/polymer composite having electromagnetic interference shielding effectiveness, which contains 0.1-10% of modified carbon nanotubes, based on the weight of the polymer. The present invention further provides methods for preparing the modified carbon nanotubes and the composite.

### CROSS-REFERENCE TO RELATED APPLICATION

The present application is a continuation-in-part application of U.S. patent application Ser. No. 12/081,517, filed Apr. 17, 2008. The above-listed application is commonly assigned with the present invention and the entire contents of which are incorporated herein by reference.

### FIELD OF THE INVENTION

The present invention provides modified carbon nanotube (CNT), and a CNT/Polymer composite having electromagnetic interference shielding effectiveness, and preparation methods thereof.

### BACKGROUND OF THE INVENTION

USP 2007/012900 A1 discloses a particulate conductive filler which comprises a conductive metal coating formed over a coarse carbon-based core such as graphite between 350 and 1000 microns in size. The conductive filler is used in conjunction with a polymer matrix such as an elastomer typified by silicone elastomer to form composite materials for conductive and electromagnetic interference shielding applications.

WO 2007/010517 A1 discloses modified polymers which are prepared by providing a nanotube or nanoparticle suspension, adding a preformed polymer, swelling the preformed polymer in the suspension, and isolating the modified polymer from the suspension. The polymer may be a swellable polymer in the form of polymeric yarns, fibres, fabrics, ribbons or films. The swelling may be carried out using ultrasonic treatment. Carbon nanotubes, magnetic (Fe3O4) and fluorescent (CdTe) nanoparticles suspensions have been utilized to demonstrate the fabrication of new polymer composites. The magnetic polymer composites are useful in electromagnetic interference (EMI) shielding of medical equipment in hospitals, computers and consumer electronics.

USP 2005/127329 A1 discloses a method of reinforcing a polymeric material with nanosize materials, in which materials such as vapor grown carbon nanofibers, carbon nanotubes, layered silicates, nanosize sphered silica, or graphite nanoparticles are combined with a polymer and a solvent to form a substantially homogeneous mixture, followed by removal of the solvent by evaporation or coagulation. Depending on the nanosize materials used, the resulting polymeric nanocomposite material exhibits high electrical and thermal conductivity, enhanced mechanical strength, abrasion resistance, reduced gas permeation, and/or dimensional stability. The polymeric nanocomposite material may be used in electromagnetic interference shielding. The polymer used in this prior art invention is preferably selected from the group consisting of polyurethanes, polyolefins, polyamides, polyimides, epoxy resins, silicone resins, polycarbonate resins, acrylic resins, and aromatic-heterocyclic rigid-rod and ladder polymers.

WO 2004/097853 A1 discloses a conductive carbon nanotube-polymer composite comprising carbon nanotubes and a polymer, wherein the carbon nanotubes primarily reside between coalesced particles of the polymer. The composite is prepared with a suspension of carbon nanotubes that can be stabilized with a stabilizer, such as a water-soluble polymer or surfactant. The nanotube suspension is mixed with a polymer suspension of polymer particles that substantially exclude the nanotubes. The polymer suspension can be stabilized with a stabilizer, such as a water-soluble polymer or surfactant. After mixing the two suspensions, water and any solvent are removed to form a nanotube-polymer composite. As liquid is removed from the nanotube-polymer suspension, the polymer particles coalesce and the nanotubes become trapped and aggregate primarily between the polymer particles, wherein the nanotubes form a conductive network in the polymer composite. Electrical percolation was realized with less than 0.04 wt % single-wall carbon nanotubes in poly(vinyl acetate).

The inventors of the present invention disclose in U.S. patent application Ser. No. 12/081,517 (publication No. US 2009-0104361 A1) a method of preparing carbon nanotube/polymer composite having electromagnetic interference (EMI) shielding effectiveness, which comprises dispersing multi-walled carbon nanotubes (MWCNT) in an organic solvent such as N,N-Dimethylacetamide (DMAc); dissolving monomers such as methyl methacrylate (MMA) and an initiator such as 2,2-azobisisobutyronitrile (AIBN) in the MWCNT dispersion; and polymerizing the monomers in the resulting mixture at an elevated temperature such as 120° C. to form a MWCNT/PMMA composite. The composite is coated onto a PET film, and the coated PET film alone or a stack of multiple coated PET films can be applied as an EMI shielding material.

Friedel-Crafts acylation is a well known reaction, details of which can be found on http://en.wikipedia.org/wiki/Friedel-Crafts_reaction.

### SUMMARY OF THE INVENTION

An objective of the present invention is to provide a modified CNT.

Another objective of the present invention is to provide a modified CNT/polymer composite having electromagnetic interference (EMI) shielding effectiveness.

A further objective of the present invention is to provide a method of preparing a modified CNT.

Another further objective of the present invention is to provide a method of preparing modified CNT/polymer composite having electromagnetic interference (EMI) shielding effectiveness.

In order to accomplish the aforesaid objective a modified carbon nanotube (CNT) prepared in accordance of the present invention comprises a CNT and —C(O)—R′ or —C(O)—R—COOH covalently bounded to a surface of said CNT, wherein R′ is C1-C26 alkyl or C2-C26 alkenyl, and R is C1-C26 alkylene or C2-C26 alkenylene.

Preferably, the modified CNT has —C(O)—R—COOH covalently bounded to the surface thereof. More preferably, R is is —C═C—.

The present invention also discloses a method for preparing a modified carbon nanotube (CNT) comprising undergoing Friedel-Crafts acylation of a CNT and an acid anhydride or acyl chloride in a solvent, in the presence of a catalyst, in an inert atmosphere and under refluxing.

Preferably, said method comprises undergoing Friedel-Crafts acylation of the CNT and the acid anhydride acid. More preferably, said acid anhydride is (RCO)₂O, and R is C1-C26 alkylene or C2-C26 alkenylene. Most preferably, R is —C═C—.

The present invention further discloses a modified CNT/polymer composite having electromagnetic interference shielding effectiveness, which comprises a polymer and 0.1-10% of the modified CNT of the present invention dispersed therein, based on the weight of the polymer.

Preferably, the composite is a layer having a thickness of 0.05 mm to 1.0 mm formed on a substrate.

Preferably, said CNT is a single-walled or multi-walled CNT (MWCNT).

Preferably, said CNT is a bamboo-type or a spiral-type CNT.

Preferably, said polymer is a homopolymer of monomers selected from the group consisting of acrylic acid, methacrylic acid, methyl acrylate, methyl methacrylate, and styrene; or a copolymer of acrylonitrile, butadiene and styrene. More preferably, said polymer is poly(methyl methacrylate).

Preferably, said polymer is poly(acrylic acid), poly(methacrylic acid), poly(methyl acrylate), poly(methyl methacrylate), polystyrene, soluble polyamide, soluble polyamideimide, polyamide, soluble polyurethane, unsaturated polyester, acrylonitrile-butadiene-styrene copolymer, poly-ether-sulfone (PES), soluble poly-ether-imide (PEI), poly(vinyl ester), thermoplastic polyurethane, silicone or epoxy resin.

The present invention further discloses a method for preparing a modified CNT/polymer composite having electromagnetic interference (EMI) shielding effectiveness, comprising the following steps:

a) preparing a polymer solution containing 0.1-10 wt % of the modified carbon nanotubes of the present invention dispersed therein, based on the weight of the polymer; and b) coating the polymer solution containing the modified carbon nanotubes dispersed therein on a substrate and drying the resulting layer coated on the substrate.

Preferably, the method of the present invention further comprises the following step: c) stacking a plurality of the substrates prepared from step b), each of which has the dried layer. More preferably, step c) further comprises applying an adhesive on the substrates prior to said stacking so that the stacked substrates are bonded.

Preferably, the dried layer on the substrate prepared in step b) has a thickness of 0.05 mm to 1.0 mm. More preferably, 2 to 100 sheets of the substrates are stacked in step c).

Preferably, said preparing in step a) comprises dispersing the modified carbon nanotubes in an organic solvent; dissolving monomers and an initiator in the carbon nanotubes dispersion; and polymerizing the monomers in the resulting mixture to form said polymer solution containing the modified carbon nanotubes dispersed therein. Said monomers preferably are selected from the group consisting of acrylic acid, methacrylic acid, methyl acrylate, methyl methacrylate, and styrene; or said monomers are a mixture of acrylonitrile, butadiene and styrene. Among them, methyl methacrylate (MMA) is more preferable. Said organic solvent preferably is N,N-Dimethylacetamide (DMAc), said initiator preferably is 2,2-azobisisobutyronitrile (AIBN), and said monomers preferably are polymerized at 120° C.

Alternatively, said preparing in step a) comprises dissolving a polymer in an organic solvent and dispersing the modified carbon nanotubes in the resulting polymer solution to form said polymer solution containing carbon nanotubes dispersed therein. Said polymer preferably is selected from the group consisting of poly(acrylic acid), poly(methacrylic acid), poly(methyl acrylate), poly(methyl methacrylate), soluble polyimide, soluble poly(amide imide), polyamide, polystyrene, soluble polyurethane, unsaturated polyester, poly(ether sulfone), soluble poly(ether imide), poly(vinyl ester), thermoplastic polyurethane, silicone, and epoxy resin. Among them poly(methyl methacrylate) (PMMA) is more preferable. Said PMMA is preferably prepared by polymerizing methyl methacrylate in a solvent of N,N-Dimethylacetamide and in the presence of an initiator of 2,2-azobisisobutyronitrile at 120° C.

Preferably, said substrate in step b) is a film of poly(ethylene terephthalate), polyimide, polyethylene, polypropylene, or poly(vinyl chloride). More preferably, said substrate in step b) is a poly(ethylene terephthalate) (PET) film.

Preferably, said substrate in step b) is an insulation layer enclosing an electric wire.

### BRIEF DESCRIPTION OF THE DRAWINGS

FIG. 1 is a Raman spectrum of pristine MWCNTs used in the present invention.

FIG. 2 is a Raman spectrum of the modified MWCNTs prepared in Preparative Example 1 of the present application.

FIG. 3 shows C 1s core-level XPS spectra of pristine MWCNTs used in the present invention.

FIG. 4 shows C 1s core-level XPS spectra of the modified MWCNTs prepared in Preparative Example 1 of the present application.

FIG. 5 is a plot showing EMI shielding effectiveness (2-18 GHz) of one to ten layers of 1.0 mm MWCNT/PMMA composite sheets prepared in Control Example 1 of the present application.

FIG. 6 is a plot showing EMI shielding effectiveness (2-18 GHz) of one to ten layers of 1.0 mm MWCNT/PMMA composite sheets prepared in Control Example 2 of the present application.

FIG. 7 is a plot showing EMI shielding effectiveness (2-18 GHz) of one to ten layers of 1.0 mm MWCNT/PMMA composite sheets prepared in Example 1 of the present invention.

FIG. 8 is a plot showing EMI shielding effectiveness (2-18 GHz) of one to ten layers of 1.0 mm MWCNT/PMMA composite sheets prepared in Example 2 of the present invention.

FIG. 9 is a plot showing the wear endurance test results of specimens prepared in Control Examples 3 and 4, and Examples 3 and 4 of the present invention.

### DETAILED DESCRIPTION OF THE INVENTION

A carbon nanotube/polymer composite having electromagnetic interference (EMI) shielding effectiveness prepared by a method according to one of the preferred embodiments of the present invention comprises the following steps:
a) Dispersing modified carbon nanotubes (Mah-g-MWCNT) in DMAc;
b) Adding MMA and AIBN to the Mah-g-MWCNT dispersion prepared in Step (a);
c) Polymerizing MMA at 120° C. to form a solution of Mah-g-MWCNT/PMMA composite;
d) Coating the solution from Step c) on a PET film and drying the solution coated on the PET film; and
e) Stacking a plurality of coated PET films from Step d) and using the stacked PET films as an EMI shielding material.

A carbon nanotube/polymer composite having electromagnetic interference (EMI) shielding effectiveness prepared by a method according to another preferred embodiment of the present invention comprises the following steps:
A) Dissolving MMA and AIBN in DMAc;
B) Polymerizing MMA at 120° C. to form a solution of PMMA;
C) Adding Mah-g-MWCNT to the PMMA solution prepared in Step (B) and dispersing Mah-g-MWCNT in the PMMA solution under ultrasonication;
D) Coating the PMMA solution containing Mah-g-MWCNT dispersed therein from Step C) on a PET film and drying the solution coated on the PET film; and
   E) Stacking a plurality of coated PET films from Step d) and using the stacked PET films as an EMI shielding material.

The present invention will be better understood through the following Examples, which are merely for illustration and not for limiting the scope of the present invention. Materials used in the following Examples and Control Examples were:
Multi-Walled CNT (abbreviated as P-MWCNT) produced by The CNT Company, Inchon, Korea. This type of CNT was prepared by a CVD process. The CNTs had a purity of 93%, a diameter of 10-50 nm, a length of 1-25 μm, and a specific surface area of 150-250 m²g⁻¹.
Methyl methacrylate (MMA) manufactured by Acros Organics Co., New Jersey, USA.
2,2-azobisisobutyronitrile (AIBN) manufactured by Tokyo Chemical Industry Co., Ltd., Tokyo, Japan.

### Preparative Example 1

### MWCNTs Modified by Friedel-Crafts Acylation

5 g of pristine multi-walled carbon nanotubes (P-MWCNTs), 500 mL of nitro-methane and 50 g of anhydrous maleic acid anhydride were introduced into a three-neck flask, where a ultrasonication was carried out for one hour and half to dissolve the anhydrous maleic acid anhydride in nitor-methane completely and well disperse P-MWCNTs in the solution. 140 g of AlCl₃ was added to the dispersion, and the flask was vacuumed and nitrogen was then introduced. In nitrogen atmosphere the mixture was reacted at 60° C. under refluxing for 60 hours.

500 mL of distilled water was added to the reaction mixture after the reaction, which was then subjected to a ultrasonication for one hour, so that unreacted AlCl₃ was dissolved in the distilled water. The resulting mixture was then filtered with suction to obtain powder, which was then added to 500 mL HCl, and ultrasonicated for one hour to neutralize the unreacted maleic acid anhydride on the powder, followed by filtration with suction. The resulting powder was washed with distilled water, and dried in vacuo to obtain a final product of carbon nanotubes modified by Friedel-Crafts acylation (Mah-g-MWCNT), which were identified by XPS and Raman spectra.

### Identification of Modified MWCNT by Raman Spectrum

Raman spectra were recorded with LabRam I confocal Raman spectrometer (Dilor, France). The excitation wavelength was 632.8 nm from a He—Ne laser with a laser power of ca. 15 mW at the sample surface. A holographic notch filter reflected the exciting line into an Olympus BX40 microscope (Tokyo, Japan). X-Ray photoelectron spectra (XPS) measurements were performed using a VG Scientific ESCALAB 220 iXL spectrometer equipped with a hemispherical electron analyzer and an Mg Ka (hν=1487.7 eV) X-ray source. A small spot lens system allowed analysis of a sample that was less than 1 mm² in area.

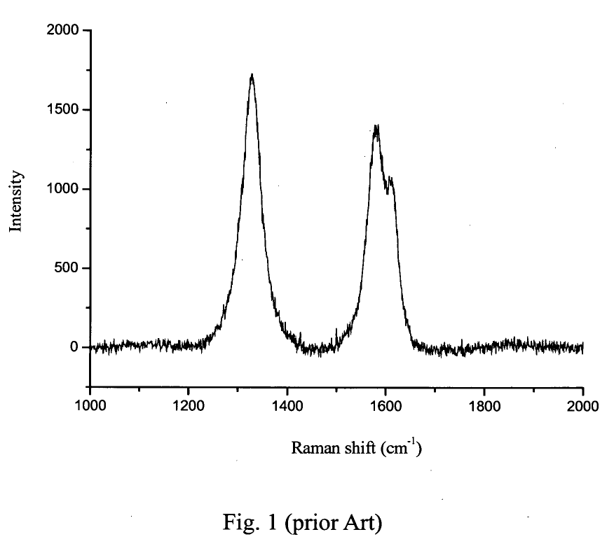

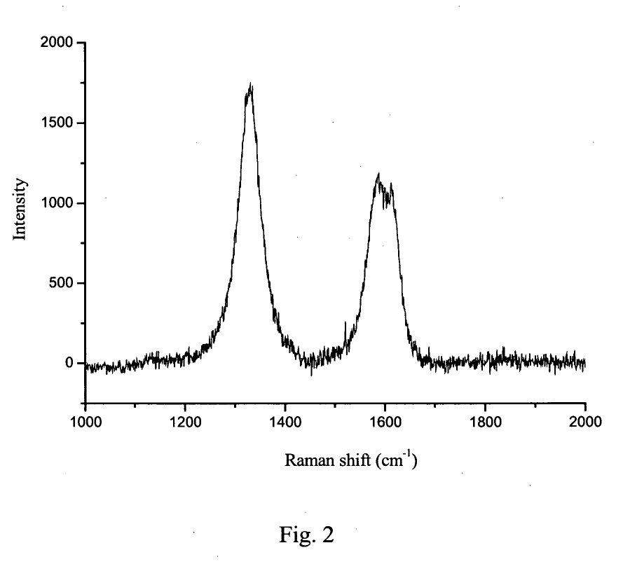

Raman spectroscopy is a powerful tool to investigate the extent of disorder in the functionalized MWCNT. FIG. 1 and FIG. 2 present the Raman spectra of pristine MWCNT (P-MWCNT) and modified MWCNT (Mah-g-MWCNT), respectively. The D- and G-bands at ˜1322 cm⁻¹ and ˜1570 cm⁻¹, respectively, attributed to defects/disorder-induced modes (or sp³-hybridized carbons) and in-plane vibrations of the graphite wall (or sp²-hybridized carbons), are clearly observable for functionalized MWCNT. The extent of defects in graphite materials upon surface modification can be quantified by the area ratio of D- to G-bands (i.e. Dₐ/Gₐ). The Dₐ/Gₐ area ratio of the pristine MWCNTs is ca. 1.08. For the modified MWCNT, the Dₐ/Gₐ area ratio is ˜1.292, showing increased Dₐ/Gₐ values compared with pristine MWCNTs. Therefore, the increase in Dₐ/Gₐ area ratio after modification reveals the formation of defects or functional group on the surfaces of MWCNT due to covalently grafting of maleic acid anhydride onto the surfaces of MWCNT.

### High-Resolution C 1s Core-level X-ray Photoelectron Spectroscopy (XPS) Spectra of the Surface of the Pristine MWCNT and Modified MWCNT

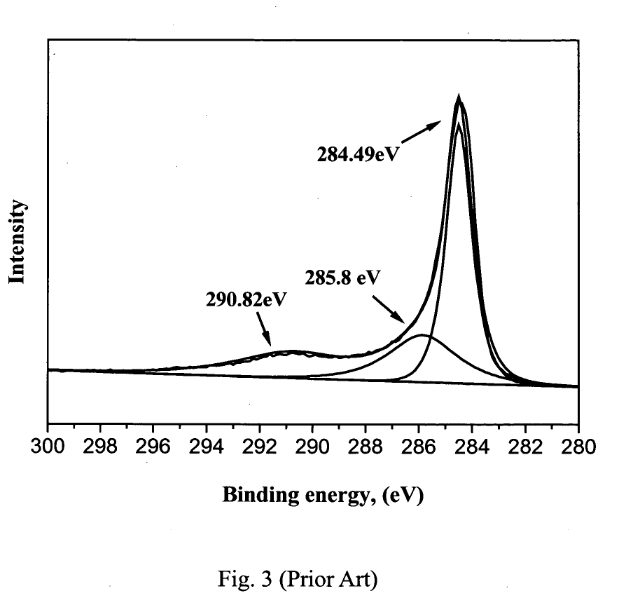

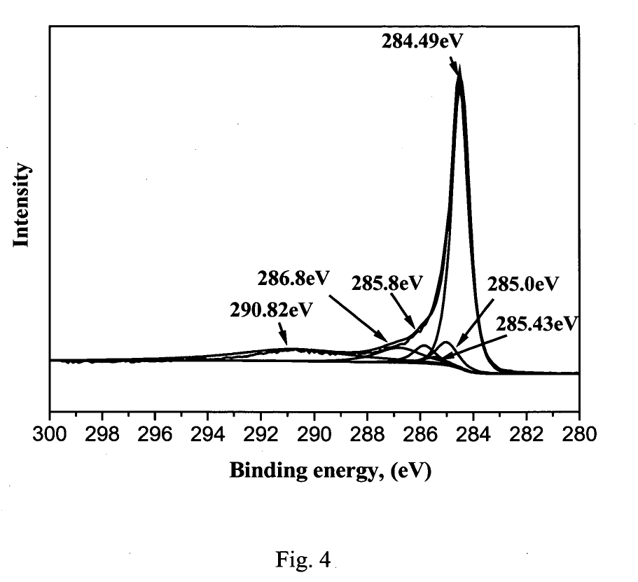

FIGS. 3 and 4 show the C 1s core-level XPS spectra of pristine MWCNT (P-MWCNT) and modified MWCNT (Mah-g-MWCNT), respectively, which further confirm the covalently grafting of maleic acid anhydride onto the surfaces of MWCNT. Three main peaks at 284.49 eV, 285.85 eV and 290.82 eV are shown in FIG. 3, wherein 284.49 eV indicates C—H and C—C structures on the surfaces of MWCNT, 285.85 eV indicates C—O structure on the surfaces of MWCNT, and 290.82 eV indicates —COO structure on the surfaces of MWCNT. There are three additional peaks shown in FIG. 4, which are at 285.00 eV, 285.43 eV and 286.8 eV. The peaks at 285.00 eV and 285.43 eV are the characteristic peak of C═C structure of the maleic acid anhydride grafted onto the surfaces of MWCNT, and 286.8 eV is attributed to C—O structure of the maleic acid anhydride grafted onto the surfaces of MWCNT. The characteristic peak of —COO of the maleic acid anhydride grafted onto the surfaces of MWCNT overlaps with that of the —COO structure on the surfaces of MWCNT. Therefore, these XPS results clearly indicate that maleic acid anhydride is covalently grafted onto the MWCNT surface.

### Thermogravimetric Analysis (TGA) of Modified MWCNT

The content of organic molecules in the modified MWCNT (Mah-g-MWCNT), was determined as the weight loss at 500° C. by TGA. The results show that the modified MWCNT (Mah-g-MWCNT) has 5.05 wt % content of organic molecules.

### Control Example 1

### In-situ Polymerization

2.62 g of P-MWCNT were dispersed in 97.5 g of DMAc; 52.5 g of MMA and 0.11 g of AIBN initiator were added into the resulting dispersion; next, the mixture was allowed to undergo polymerization at 120° C. under refluxing for four hours, so that a solution of MWCNT/PMMA composite having 4.76 wt % of MWCNT was formed. The MWCNT/PMMA composite solution was coated on a PET film, and the resulting coating layer was dried by evaporating the organic solvent therefrom to form a MWCNT/PMMA composite layer having a thickness of 0.1 cm on the PET film. The coating area was 20 cm×20 cm. 10 sheets of the coated PET films were prepared as above. A single sheet of the coated PET film was used as an EMI shielding material, or multiple sheets of the coated PET films were stacked and the resulting stack was used as an EMI shielding material.

### Control Example 2

### Ex-situ Polymerization

52.5 g of MMA and 0.11 g of AIBN initiator were dissolved in 97.5 g of DMAc, and the solution was allowed to undergo polymerization at 120° C. under refluxing for four hours to form a PMMA solution. 2.62 g of P-MWCNT was dispersed in the PMMA solution under ultrasonication. The MWCNT/PMMA composite solution was coated on a PET film, and the resulting coating layer was dried by evaporating the organic solvent therefrom to form a MWCNT/PMMA composite layer having a thickness of 0.1 cm on the PET film. The coating area was 20 cm×20 cm. 10 sheets of the coated PET films were prepared as above. A single sheet of the coated PET film was used as an EMI shielding material, or multiple sheets of the coated PET films were stacked and the resulting stack was used as an EMI shielding material.

### Example 1

### In-situ Polymerization

The procedures in Control Example 1 were repeated except that the pristine MWCNT (P-MWCNT) was replaced by the modified MWCNT (Mah-g-MWCNT) prepared in Preparative Example 1.

### Example 2

### Ex-situ Polymerization

The procedures in Control Example 2 were repeated except that the pristine MWCNT (P-MWCNT) was replaced by the modified MWCNT (Mah-g-MWCNT) prepared in Preparative Example 1.

EMI shielding effectiveness (2-18 GHz) was measured by using a HP 8722ES Vector Network Analyzer manufactured by Damaskos, Inc., Concordville, Pa., USA.

### Results:

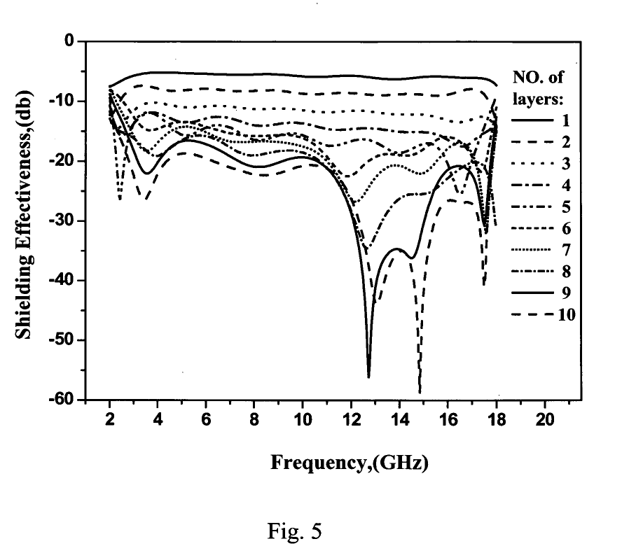

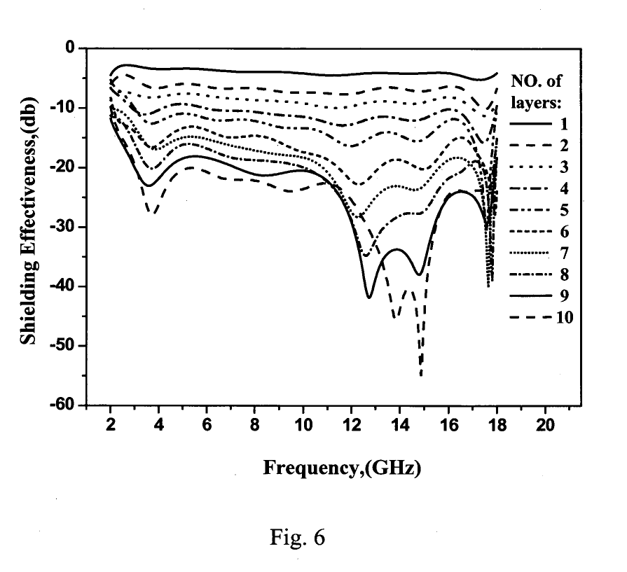

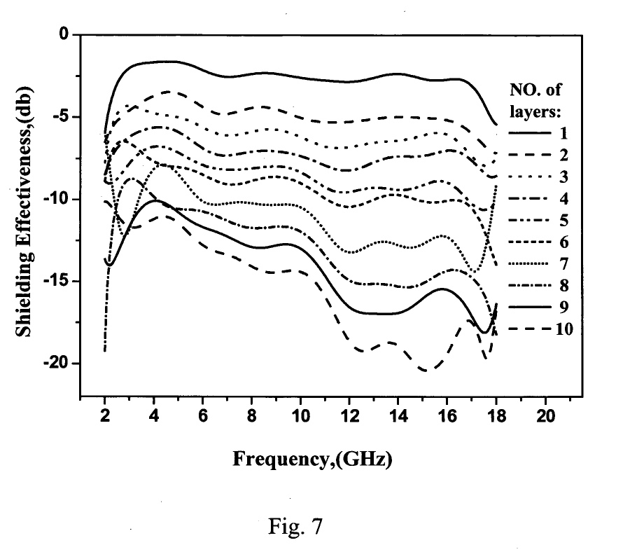

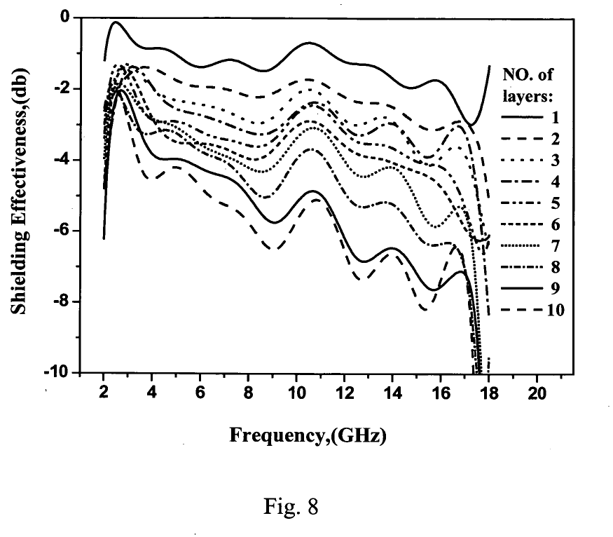

FIGS. 5 to 8 shows the EMI shielding effectiveness (2-18 GHz) of the composite coated PET sheets prepared in Control Examples 1 to 2 and Examples 1 to 2, respectively. It can be seen from FIGS. 5 and 6 or FIGS. 7 and 8 that the coated PET film or the stacks of multiple films with in-situ polymerization have better EMI shielding effectiveness in comparison with those with ex-situ polymerization. It is believed that this is a result of better dispersion of MWCNT in the in-situ polymerization case.

The pristine MWCNT/PMMA coated PET film or the stacks of multiple films prepared in Control Examples 1 and 2 of the present invention have better EMI shielding effectiveness in comparison with the PET film(s) coated with the modified Mah-g-MWCNT/PMMA composite having the same MWCNT contents prepared in Examples 1 and 2, as shown in FIGS. 5 and 7 or FIGS. 6 and 8. It is reckoned that the pristine MWCNT retains more metal catalyst originally used for forming the MWCNT than the modified MWCNT because the latter has been subjected to acid washing during the preparation thereof.

### Control Example 3

### In-situ Polymerization

The P-MWCNT/PMMA composite prepared in Control Example 1 was poured into a PET mold to prepare a sheet of 20 cm×20 cm having a thickness of 1 mm (the P-MWCNT content of 4.76 wt %). A circular specimen for wear endurance test having a diameter of 18 cm and a central hole of 8 mm was cut from the sheet.

The procedures in Control Example 1 were repeated by varying the amount of P-MWCNT used to prepare P-MWCNT/PMMA composites having 0.25, 0.50, 0.74, 0.99 and 2.44 wt % of P-MWCNT, which were then used to prepare circular specimens for wear endurance test as above.

### Control Example 4

### Ex-situ Polymerization

The P-MWCNT/PMMA composite prepared in Control Example 2 was poured into a PET mold to prepare a sheet of 20 cm×20 cm having a thickness of 1 mm (the P-MWCNT content of 4.76 wt %). A circular specimen for wear endurance test having a diameter of 18 cm and a central hole of 8 mm was cut from the sheet.

The procedures in Control Example 2 were repeated by varying the amount of P-MWCNT used to prepare P-MWCNT/PMMA composites having 0.25, 0.50, 0.74, 0.99 and 2.44 wt % of P-MWCNT, which were then used to prepare circular specimens for wear endurance test as above.

### Example 3

### In-situ Polymerization

The Mah-g-MWCNT/PMMA composite prepared in Example 1 was poured into a PET mold to prepare a sheet of 20 cm×20 cm having a thickness of 1 mm (the P-MWCNT content of 4.76 wt %). A circular specimen for wear endurance test having a diameter of 18 cm and a central hole of 8 mm was cut from the sheet.

The procedures in Example 1 were repeated by varying the amount of Mah-g-MWCNT used to prepare Mah-g-MWCNT/PMMA composites having 0.25, 0.50, 0.74, 0.99 and 2.44 wt % of Mah-g-MWCNT, which were then used to prepare circular specimens for wear endurance test as above.

### Example 4

### Ex-situ Polymerization

The Mah-g-MWCNT/PMMA composite prepared in Example 2 was poured into a PET mold to prepare a sheet of 20 cm×20 cm having a thickness of 1 mm (the P-MWCNT content of 4.76 wt %). A circular specimen for wear endurance test having a diameter of 18 cm and a central hole of 8 mm was cut from the sheet.

The procedures in Example 2 were repeated by varying the amount of Mah-g-MWCNT used to prepare Mah-g-MWCNT/PMMA composites having 0.25, 0.50, 0.74, 0.99 and 2.44 wt % of Mah-g-MWCNT, which were then used to prepare circular specimens for wear endurance test as above.

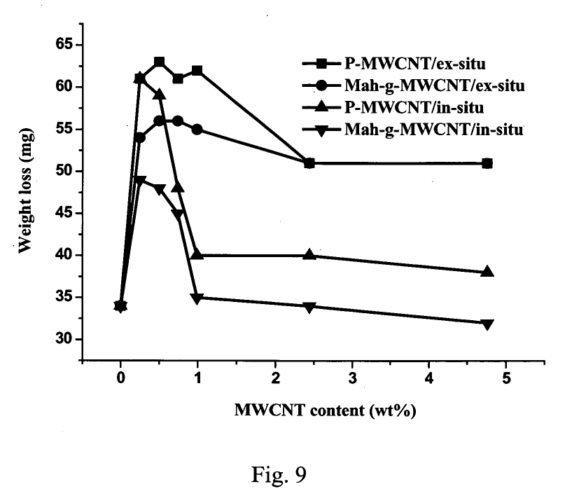

An objective of the present invention is to prepare a coating for EMI shielding, and thus it makes the surface wear endurance of the composite of the present invention become am important property. Wear endurance test for the composite specimens was conducted by using TABER QC-619T, Silitech Technology Co., Taiwan, with a weight load of 500 g and a speed of 60 rpm for 10 minutes. The results are shown in FIG. 9.

It can be observed from FIG. 9 that little addition of MWCNT to PMMA will cause the weight wore off increase in most cases. This might be contributed to the dimension of wearing off by the sand wheel being greater than nano scale of MWCNT. As a result, the wear endurance of the composite cannot be enhanced when the content of MWCNT is too low. However, the weight wore off starts decreasing when the MWCNT content is greater than 0.5 wt %. This is because MWCNT content of 0.5 wt % is higher than the percolation threshold, and MWCNT will form a network structure in the composite. The binding between MWCNT and PMMA matrix is enhanced significantly, so that the wear endurance of the composite is improved. In comparison with the composites prepared by in-situ polymerization and ex-situ polymerization, the former has a better wear endurance than the latter. Thanks to the in-situ polymerization more PMMA molecules grafted onto the surfaces of MWCNTs, which in turn causes a better dispersion of MWCNT in and adhesion of MWCNT to PMMA matrix. This is more prominent for the composites containing modified MWCNT (Mah-g-MWCNT).

The specimens after wear endurance tests were observed with SEM, and the surface roughness thereof observed shows that the composites prepared by in-situ polymerization have a more smoother surface and a less degree of destruction than the composites prepared by ex-situ polymerization. This observation is consistent with the results of the above wear endurance test.

1. A modified carbon nanotube (CNT) comprising a CNT and —C(O)—R′ or —C(O)—R—COOH covalently bounded to a surface of said CNT, wherein R′ is C1-C26 alkyl or C2-C26 alkenyl, and R is C1-C26 alkylene or C2-C26 alkenylene.

2. The modified CNT as claimed in claim 1, wherein the modified CNT has —C(O)—R—COOH covalently bounded to the surface thereof.

3. The modified CNT as claimed in claim 2, wherein R is —C═C—.

4. A method for preparing a modified carbon nanotube (CNT) comprising undergoing Friedel-Crafts acylation of a CNT and an acid anhydride or acyl chloride in a solvent, in the presence of a catalyst, in an inert atmosphere and under refluxing.

5. The method as claimed in claim 4 comprising undergoing Friedel-Crafts acylation of the CNT and the acid anhydride acid.

6. The method as claimed in claim 5, wherein said acid anhydride is (RCO)₂O, and R is C1-C26 alkylene or C2-C26 alkenylene.

7. The method as claimed in claim 6, wherein R is —C═C—.

8. A modified carbon nanotube (CNT)/polymer composite having electromagnetic interference shielding effectiveness, which comprises a polymer and 0.1-10% of the modified CNT as set forth in claim 1 dispersed therein, based on the weight of the polymer.

9. The composite as claimed in claim 8, wherein the composite is a layer having a thickness of 0.05 mm to 1.0 mm formed on a substrate.

10. The composite as claimed in claim 8, wherein said CNT is a single-walled or multi-walled CNT.

11. The composite as claimed in claim 8, wherein said CNT is a bamboo-type or a spiral-type CNT.

12. The composite as claimed in claim 8, wherein said polymer is a homopolymer of monomers selected from the group consisting of acrylic acid, methacrylic acid, methyl acrylate, methyl methacrylate, and styrene; or a copolymer of acrylonitrile, butadiene and styrene.

13. The composite as claimed in claim 12, wherein said polymer is poly(methyl methacrylate).

14. The composite as claimed in claim 12, wherein said polymer is poly(acrylic acid), poly(methacrylic acid), poly(methyl acrylate), poly(methyl methacrylate), polystyrene, soluble polyamide, soluble polyamideimide, polyamide, soluble polyurethane, unsaturated polyester, acrylonitrile-butadiene-styrene copolymer, poly-ether-sulfone (PES), soluble poly-ether-imide (PEI), poly(vinyl ester), thermoplastic polyurethane, silicone or epoxy resin.

15. The composite as claimed in claim 8, wherein the modified CNT has —C(O)—R—COOH covalently bounded to the surface thereof.

16. The composite as claimed in claim 15, wherein R is —C═C—.

17. A method of preparing carbon nanotube/polymer composite having electromagnetic interference (EMI) shielding effectiveness, which comprises the following steps: a) preparing a polymer solution containing 0.1-10 wt % of the modified CNT as set forth in claim 1 dispersed therein, based on the weight of the polymer; and b) coating the polymer solution containing the modified CNT dispersed therein on a substrate and drying the resulting layer coated on the substrate.

18. The method as claimed in claim 17 further comprising the following step: c) stacking a plurality of the substrates prepared from step b), each of which has the dried layer.

19. The method as claimed in claim 18, wherein step c) further comprises applying an adhesive on the substrates prior to said stacking so that the stacked substrates are bonded.

20. The method as claimed in claim 17, wherein the dried layer on the substrate prepared in step b) has a thickness of 0.05 mm to 1.0 mm.

21. The method as claimed in claim 19, wherein the dried layer on the substrate prepared in step b) has a thickness of 0.05 mm to 1.0 mm, and 2 to 100 sheets of the substrates are stacked in step c).

22. The method as claimed in claim 17, wherein said preparing in step a) comprises dispersing the modified CNT in an organic solvent; dissolving monomers and an initiator in the modified CNT dispersion; and polymerizing the monomers in the resulting mixture to form said polymer solution containing the modified CNT dispersed therein.

23. The method as claimed in claim 17, wherein said preparing in step a) comprises dissolving a polymer in an organic solvent and dispersing the modified CNT in the resulting polymer solution to form said polymer solution containing the modified CNT dispersed therein.

24. The method as claimed in claim 17, wherein the CNT is a single-walled or multi-walled carbon nanotube.

25. The method as claimed in claim 17, wherein the CNT is a bamboo-type or spiral-type CNT.

26. The method as claimed in claim 22, wherein said monomers are selected from the group consisting of acrylic acid, methacrylic acid, methyl acrylate, methyl methacrylate, and styrene; or said monomers are a mixture of acrylonitrile, butadiene and styrene.

27. The method as claimed in claim 26, wherein said monomers are methyl methacrylate.

28. The method as claimed in claim 27, wherein said organic solvent is N,N-Dimethylacetamide, said initiator is 2,2-azobisisobutyronitrile, and said monomers are polymerized at 120° C.

29. The method as claimed in claim 23, wherein said polymer is selected from the group consisting of poly(acrylic acid), poly(methacrylic acid), poly(methyl acrylate), poly(methyl methacrylate), soluble polyimide, soluble poly(amide imide), polyamide, polystyrene, soluble polyurethane, unsaturated polyester, poly(ether sulfone), soluble poly(ether imide), poly(vinyl ester), thermoplastic polyurethane, silicone, and epoxy resin.

30. The method as claimed in claim 29, wherein said polymer is poly(methyl methacrylate).

31. The method as claimed in claim 30, wherein said poly(methyl methacrylate) is prepared by polymerizing methyl methacrylate in a solvent of N,N-Dimethylacetamide and in the presence of an initiator of 2,2-azobisisobutyronitrile at 120° C.

32. The method as claimed in claim 17, wherein said substrate in step b) is a film of poly(ethylene terephthalate), polyimide, polyethylene, polypropylene, or poly(vinyl chloride).

33. The method as claimed in claim 32, wherein said substrate in step b) is a poly(ethylene terephthalate) film.

34. The method as claimed in claim 21, wherein said substrate is an insulation layer enclosing an electric wire.

35. The method as claimed in claim 17, wherein the modified CNT has —C(O)—R—COOH covalently bounded to the surface thereof.

36. The method as claimed in claim 35, wherein R is —C═C—.
